(19) United States
(12) Patent Application Publication
(10) Pub. No.: US 2009/0098585 A1
(43) Pub. Date: Apr. 16, 2009

(54) REAGENT KIT FOR DETECTING LUPUS ANTICOAGULANT

(75) Inventor: Masahiro OKUDA, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/269,719

(22) Filed: Nov. 12, 2008

Related U.S. Application Data
(62) Division of application No. 10/622,736, filed on Jul. 21, 2003, now abandoned.

(30) Foreign Application Priority Data
Jul. 25, 2002 (JP) 2002-216782

(51) Int. Cl. C12Q 1/56

(52) U.S. Cl. 435/13

(57) ABSTRACT

A reagent kit capable of distinguishing between a blood sample containing lupus anticoagulant and blood samples from individuals having other anticoagulant diseases is disclosed. The reagent kit comprises two coagulation time reagents containing phosphatidylserine in a different phosphatidylserine content ratio to the total content of phospholipids from each other, threreby giving different coagulation times with use of corresponding coagulation time reagent.

This is a divisional of Application Ser. No. 10/622,736 filed Jul. 21, 2003. The entire disclosure(s) of the prior application, application Ser. No. 10/622,736 is hereby incorporated by reference.

### BACKGROUND OF THE INVENTION

1. Field of the Invention

This invention relates to a blood coagulation reagent kit used in diagnosis of anti-phospholipid (aPL) antibody syndrome in the fields such as a clinical chemistry and a medical study, and more particularly pertains to a reagent kit capable of specifically detecting lupus-anticoagulant-positive diseases based on a blood test in vitro.

2. Discussion of the Related Art

Lupus anticoagulant (hereinafter, called as “LA”) is an immunoglobulin which has been found firstly in patients suffering from systemic lupus erythematosus (hereinafter, called as “SLE patients”). While LA is detected in SLE patients with a frequency ranging from 5 to 10%, it is also detected in patients suffering from other autoimmune diseases and tumorous diseases. LA is most frequently detected in patients with anti-phospholipid syndrome (APS) such as patients with thrombosis, women who have undergone miscarriage or premature labor, and patients with thrombocytopenia. Since a time required in blood coagulation with respect to patients with APS is prolonged owing to phospholipid-dependent coagulation reaction, it is conceived that LA is an auto-antibody against phospholipid.

According to a recent study, it has been elucidated that LA is a heterogeneous immunoglobulin which shows antibody reaction against a complex of negatively-charged phospholipid and β2-glycoprotein I (β2-GPI) or a complex of negatively-charged phospholipid and prothrombin. It is conceived that LA causes anti-coagulating action because bonding of LA to the complex hinders conversion of prothrombin to thrombin.

There have been known reagents containing a certain amount of phospholipid for detecting LA having the aforementioned properties such as cephalin derived from rabbit brain, cephalin derived from bovine brain, soy bean lecithin, and viper venom. If LA exists in a blood sample, the existing LA neutralizes the phospholipid in the reagent, which gives rise to shortage of the phospholipid necessary for initiating cascade reaction of coagulating the blood. As a result of such shortage, the coagulation time of each of the blood samples is prolonged in proportion to the amount of phospholipid in the reagent which has been neutralized by LA. Thus, this approach makes it possible to determine whether each blood sample is LA positive or not.

In addition to the above approach, there has been conducted another approach in which two or more different steps are combined each other in order to secure detection accuracy, including diluting the reagent in light of the fact that the less the phospholipid in the reagent, the higher the detection sensitivity of LA is.

As an example of the latter approach, there is known a technique of judging whether a blood sample is LA-positive or not by using an activated partial thromboplastin time reagent (hereinafter, simply called as “APTT reagent”), which is a combination of two kinds of reagents having different concentrations of phospholipid from each other, and by calculating Rosner Index or Lupus Ratio (LR) based on the coagulation times respectively obtained with use of the two kinds of reagents.

One of the examples of commercially available detecting reagents is Gradipore LA (manufactured by Gradipore Ltd., Australia), which is a reagent kit of measuring coagulation times with use of Russel's viper venom. The reagent kit is a combination of a first coagulation time reagent containing viper venom and a second coagulation time reagent containing viper venom and soy- bean-derived phospholipid in an excessive amount. With use of this reagent kit, a blood sample is judged to be LA positive if the following formula is satisfied:
the ratio of the coagulation time of a blood sample added with the first coagulation time reagent to that of a blood sample added with the second coagulation time reagent ≧1.3.

Further, according to a reported scientific research (see V.Chantarangkul, et al., Thromb. Res. 1992, 67:355-365; E. Rosner, et al., Thromb. Haemost. 1987, 57:144-149), there has been established a technique in which LA is arithmetically detected by utilizing a difference in concentration of phospholipid derived from rabbit brain or its equivalent.

A still another approach is proposed. According to this approach, coagulation times are measured by using an APTT reagent kit consisting of two kinds of coagulation time reagents having different phospholipid concentrations from each other or using a diluted Russel's viper venom time (dRVVT) reagent kit with use of a sample, which is a mixture of blood plasma (normal plasma) from a normal healthy individual and blood plasma (LA-containing plasma) from an LA-positive patient in mixing ratio of 1:1, and LA is detected based on a degree of correction in correcting a deviation of the coagulation time of the LA-containing plasma from the coagulation time of the normal plasma, as an index.

In the conventional approaches of measuring the degree of phospholipid-dependent coagulation, what is used as phospholipid is the one derived from natural resources, and the composition of the phospholipid is uncontrolled. Accordingly, it is highly likely that non-uniformity of natural-resource-derived phospholipid in composition or a variation in conditions of extracting the phospholipid may adversely affect the coagulation time of a blood sample with the result that the detection sensitivity of LA is fluctuated. Further, according to the conventional measurement method, it is possible to normalize the coagulation time by addition of normal blood to a blood sample from an individual having a coagulation factor deficiency because the deficient coagulation factor is supplied from the normal blood plasma. In this case, however, it is difficult to discriminate a blood sample containing a coagulation inhibitor such as warfarin or heparin from a blood sample of an LA-positive patient. Accordingly, there is likelihood that the blood samples containing these coagulation inhibitors are misjudged as LA-positive.

In view of the above, it is necessary to discriminate a prolonged coagulation time resulting from a coagulation inhibitor or a coagulation factor deficiency from a prolonged coagulation time resulting from LA in order to precisely determine disorders resulting from LA.

### SUMMARY OF THE UNVENTION

An object of this invention is to provide a reagent kit capable of specifically and precisely detecting LA by enhancing the detection sensitivity of LA and by discriminating between a blood sample from LA-positive individual and a blood sample of an individual having a coagulation factor deficiency or a blood sample containing a coagulation inhibitor such as heparin.

As a result of extensive research and study to attain the above object, the inventor found that LA-containing blood samples show a remarkable difference in coagulation time between the cases of using two kinds of reagents having a difference solely in the content of phosphatidylserine to phospholipid in the respective reagents, and thus made this invention.

In accordance with the present invention, there is provided a reagent kit for detecting LA in blood which comprises a first coagulation time reagent containing phospholipids including phosphatidylserine and a second coagulation time reagent containing phospholipids including phosphatidylserine. In this reagent kit, the content of phosphatidylserine to the total content of the phospholipids in the first coagulation time reagent is different from the content of phosphatidylserine to the total content of the phospholipids in the second coagulation time reagent.

### DETAILED DESCRIPTION OF THE INVENTION

A reagent kit for detecting lupus anticoagulant (hereinafter, simply called as “LA reagent kit”) of this invention essentially consists of a first coagulation time reagent containing phospholipids including phosphatidylserine, and a second coagulation time reagent containing phospholipids including phosphatidylserine, wherein the content of the phosphatidylserine to the total content of the phospholipids in the first coagulation time reagent is different from the content of the phosphatidylserine to the total content of the phospholipids in the second coagulation time reagent.

Phosphatidylserine (hereinafter sometimes called as “PS”) contained in the first coagulation time reagent and in the second coagulation time reagent may be a synthetic phosphatidylserine or a phosphatidylserine derived from natural resources. It is preferable to use a synthetic phosphatidylserine. In case of using a natural-resource-derived phosphatidylserine, it is preferable to use phosphatidylserine of 99% or more in purity.

Preferred phospholipids contained in the first and second coagulation time reagents other than PS include phosphatidylethanolamine (hereinafter, sometimes called as “PE”) and phosphatidylcholine (hereinafter, sometimes called as “PC”). These preferred other phospholipids are necessary compositions for clot formation of a sample added with the respective first and second coagulation time reagents in vitro.

Preferably, the first (second) coagulation time reagent may contain compositions other than the above compositions that cause clot formation in vitro.

Examples of the other compositions include an activator, viper venom, calcium ions, and a tissue factor. Preferably, the activator is at least one selected from the group consisting of ellagic acid, kaolin, and celite. The viper venom is preferably at least one selected from the group consisting of Russel's viper venom, textarin venom, and ecarin venom. The tissue factor may be one derived from rabbit brain, derived from human placenta, or a recombinant.

The above other compositions are optionally selected depending on the kind of the coagulation time reagent. For instance, in case that the first (second) coagulation time reagent includes calcium ions and an activator, coagulation times are measured based on the principle of an activated partial thromboplastin time (APTT). In case that the first (second) coagulation time reagent includes calcium ions and viper venom, coagulation times are measured based on the principle of a Russel's viper venom time (RVVT). In case that the first (second) coagulation time reagent includes calcium ions and a tissue factor, coagulation times are measured based on the principle of a prothrombin time (PT).

The first (second) coagulation time reagent may contain a buffer such as HEPES and a tris buffer when need arises to do so. The concentration of the buffer solution may be in a range allowable and generally used in the field of clinical chemistry, and is determined empirically based on simplified and repeated experiments.

Specifically, the PS concentration of the first coagulation time reagent may preferably be 10 to 20 times as high as that of the second coagulation time reagent.

More specifically, the concentration of phosphatidylserine in a mixture of a sample and the first coagulation time reagent containing phospholipids including phosphatidylserine is preferably in the range from 10 to 30 μg/ml, and more preferably in the range from 15 to 20 μg/ml. On the other hand, the concentration of phosphatidylserine in a mixture of a sample and the second coagulation time reagent containing phospholipids including phosphatidylserine is preferably in the range from 1 to 7 μg/ml, and more preferably in the range from 2 to 4 μg/ml.

Alternatively, the first (second) coagulation time reagent may be a mixture of phospholipids and the other composition(s). Further alternatively, the first coagulation time reagent may consist of a first preparatory reagent and a second preparatory reagent contained in separate vessels from each other, whereas the second coagulation time reagent may consist of a third preparatory reagent and a fourth preparatory reagent contained in separate vessels from each other. In case that the first coagulation time reagent is a combination of the first and second preparatory reagents, and the second coagulation time reagent is a combination of the third and fourth preparatory reagents, the concentrations of phosphatidylserine in the first and third preparatory reagents each containing phospholipids including phosphatidylserine may be such that the concentration of phosphatidylserine in the first preparatory reagent is in the range from 3 to 1,000 μg/ml, preferably from 30 to 100 μg/ml, and more preferably from 40 to 60 μg/ml, whereas the concentration of phosphatidylserine in the third preparatory reagent is in the range from 0.2 to 200 μg/ml, preferably from 2 to 20 μg/ml, and more preferably from 6 to 10 μg/ml. It should be noted that the concentration of phosphatidylserine in the first preparatory reagent is always higher than that in the third preparatory reagent.

In case that each of the first and the third preparatory reagents contains phosphatidylethanolamine, the concentration of phosphatidylethanolamine in the first (third) preparatory reagent may be in the range from 0.1 to 300 μg/ml, preferably from 1 to 30 μg/ml, and more preferably from 3 to 15 μg/ml. In case that each of the first and the third preparatory reagents contains phosphatidylcholine, the concentration of phosphatidylcholine in the first (third) preparatory reagent may be in the range from 2 to 1,000 μg/ml, preferably from 20 to 100 μg/ml, and more preferably from 35 to 85 μg/ml.

According to this invention, in case that the inventive reagent kit is prepared based on the principle of APTT, the coagulation time reagent kit essentially consists of the first coagulation time reagent including the first preparatory reagent and the second preparatory reagent, and the second coagulation time reagent including the third preparatory reagent and the fourth preparatory reagent, wherein the first (third) preparatory reagent contains an activator and phospholipids (phosphatidylserine, phosphatidylethanolamine, and phosphatidylcholine), and the second (fourth) preparatory reagent contains calcium ions.

Further, in case that the inventive reagent kit is prepared based on the principle of RVVT, each of the first and second coagulation time reagents contains phospholipids (phosphatidylserine, phosphatidylethanolamine, and phosphatidylcholine), viper venom, and calcium ions.

Furthermore, in case that the inventive reagent kit is prepared based on the principle of PT, each of the first and second coagulation time reagents contains phospholipids (phosphatidylserine, phosphatidylethanolamine, and phosphatidylcholine), a tissue factor, and calcium ions.

The LA reagent kit of this invention has a feature that the kit is a combination of two kinds of reagents which differ in coagulation time from each other by differentiating the content of phosphatidylserine (PS content ratio) in the first coagulation time reagent to the total content of phospholipid from the PS content ratio in the second coagulation time reagent. In other words, the LA reagent kit of this invention is prepared by combining the first and second coagulation time reagents having different PS content ratios in such a manner that the coagulation times by the respective reagents are distinguishably different from each other by neutralizing PS in the first coagulation time reagent by LA.

Next, described is a method of detecting LA with use of the LA reagent kit of this invention.

First, a sample is divided into two parts, and the first coagulation time reagent containing PS in high concentration is added to one half of the sample, whereas the second coagulation time reagent containing PS in low concentration is added to the other half of the sample. In case of using the first coagulation time reagent consisting of the first and second preparatory reagents, it is possible to mix the first and second preparatory reagents together prior to adding the mixture to the sample, or alternatively add the first (second) preparatory reagent to the sample, and then the second (or first) preparatory reagent in this order. The case of using the second coagulation time reagent consisting of the third and fourth preparatory reagents is the same as the case of using the first coagulation time reagent consisting of the first and second preparatory reagents.

In case of separately adding the first and second preparatory reagents, the mixture of the sample and the preparatory reagent may be heated according to needs after adding the first (or second) preparatory reagent or before adding the second (or first) preparatory reagent. The case of separately adding the third and fourth preparatory reagents is the same as the case of separately adding the first and second preparatory reagents.

After adding the LA reagent kit to the sample, the coagulation time of the sample is measured. The measuring method of the coagulation time may be a known method for a skilled person in the art, and can be measured, for example, by using a known automated analyzer.

The sample used in the measurement is preferably blood plasma in place of blood itself, more preferably, blood plasma after removal of platelets, and furthermore preferably, a mixture of blood plasma from a patient and normal blood plasma or normal blood plasma after removal of platelets. Using a mixture containing normal blood plasma as a sample is advantageous in preventing prolongation of coagulation time resulting from a coagulation factor deficiency as well as improving detection sensitivity in a test of detecting phospholipid-dependent blood coagulation disorder. The mixing ratio of blood plasma from a patient to normal blood plasma generally ranges from 4:1 to 1:4, and preferably is 1:1.

Coagulation tests using the LA reagent kit of this invention show that the coagulation times of samples having anticoagulant disorders such as blood samples from LA-positive patients, patients having a blood coagulation factor deficiency, patients administered with warfarin, and patients administered with heparin are longer than the coagulation times of samples of normal blood. Further, there is recognized a difference in coagulation time between use of the first coagulation time reagent and use of the second coagulation time reagent with respect to the samples from LA-positive patients. Specifically, observation on coagulation tests with respect to the samples from LA-positive patients reveals that the coagulation time of the sample in use of the second coagulation time reagent containing low-concentrated PS is longer than that of the sample in use of the first coagulation time reagent containing high-concentrated PS. On the other hand, observation on coagulation tests with respect to the samples from patients having a blood coagulation factor deficiency, patients administered with warfarin, and patients administered with heparin reveals that there is not found a noticeable difference in coagulation time between use of the first coagulation reagent and use of the second coagulation time reagent. In view of these observations, LA is specifically detectable based on a coagulation time difference between use of the first coagulation reagent and use of the second coagulation reagent.

It is true that LA is detectable by judging a coagulation time difference between use of the first coagulation reagent and use of the second coagulation reagent. In order to diagnose a sample from a patient having LA disease as LA-positive with high reliance, judging from a ratio of coagulation time of a blood sample from a patient to coagulation time of a normal blood sample, for example Rosner Index (see E. Rosner, et al., Thromb. Haemast. 1987, 57:144-149) or Lupus ratio (see R. Schjetlein, et al., Thromb. Res. 1993, 69:239-250), is preferred.

In case of samples of normal blood plasma, the PS content ratio does not affect the coagulation time. Accordingly, the LR value corresponding to the ratio of the second coagulation time reagent to the first coagulation time reagent is around 1 with respect to the samples of normal blood plasma. Likewise, the LR value of each of the samples from patients having a blood coagulation factor deficiency, patients administered with warfarin, or patients administered with heparin is around 1 because there has not been recognized a great coagulation time difference due to a difference in PS concentration despite the fact that the coagulation times of these samples are longer compared to the coagulation times of the samples of normal blood. On the other hand, in case of samples of LA-positive patients, despite use of the first and second coagulation time reagents in various concentrations and combinations thereof, it is obvious that the coagulation time in use of the second coagulation time reagent is distinguishably longer than that in use of the first coagulation time reagent. Accordingly, the reagent kit of this invention is advantageous in determining LA-positive by judging whether the LR value of each of the samples is larger than a reference value.

As mentioned above, the LA reagent kit of this invention has a feature that the concentration of PS specifically bound to LA is controllably varied between the two reagents. Accordingly, the LA reagent kit of this invention makes it possible for medical personnel diagnosing LA-positive patients to recognize the coagulation time difference easily. On the other hand, the conventional reagent kit is a combination of reagents containing natural-resource-derived phospholipid which is uncontrolled in composition. Accordingly, the conventional reagent kit fails to discriminate LA-positive patients from individuals having other anticoagulant diseases. Use of the LA reagent kit of this invention is advantageous in exclusively determining LA-positive patients without likelihood that samples from individuals administered with an inhibitor such as heparin are erroneously judged as LA-positive.

### EXAMPLES

This invention will be further illustrated below by means of a number of concrete practical examples, which however do not in any way restrict the scope of the invention.

### Example

A first and a third preparatory reagents were prepared by mixing HEPES buffer, ellagic acid, TRIS buffer, phosphatidylethanolamine (PE), phosphatidylcholine (PC) and phosphatidylserine (PS). The resulting first preparatory reagent had a pH of 7.35 and respective concentrations of 50 mM of HEPS buffer, 0.1 mM of ellagic acid, 25 mM of tris buffer, 50 μg/ml of PE, 50 μg/ml of PS, and 70 μg/ ml of PC. The resulting third preparatory reagent had a pH of 7.35 and respective concentrations of 50 mM of HEPES buffer, 0.1 mM of ellagic acid, 25 mM of tris buffer, 50 μg/ml of PE, 10 μg/ml of PS, and 70 μg/ml of PC. A second and a fourth preparatory reagents each containing 25 mM/ml of calcium ions were prepared by adding CaCl₂ to purified water.

COAGUTROL N ® was used as a sample of normal plasma. A mixture of patient's plasma and the normal plasma in one-to-one ratio was used as a sample of abnormal blood plasma. As samples of abnormal plasma, four kinds of plasma samples from LA-positive patients, four kinds of plasma samples from patients administered with warfarin and a plasma sample from a patient administered with heparin were used.

Each of the samples prepared above was divided into two groups. Each sample of the one group was subjected to the treatment with the first coagulation time reagent (hereinafter called as “H-APTT reagent”), while each sample of the other group was subjected to the treatment with the second coagulation time reagent (hereinafter called as “L-APTT reagent”). The treatment with H-APTT reagent was conducted in a manner that a mixture of 50 μl of each sample and the above-prepared first preparatory reagent was heated for three minutes at 37° C. and then 50 μl of the above-prepared second preparatory reagent was added to trigger coagulation. The treatment with L-APTT reagent was conducted in the same manner as the treatment with H-APTT reagent except for use of the third and fourth preparatory reagents instead of the first and second preparatory reagents respectively.

Coagulation time, which may be measured as a time required for a certain clot formation, was measured with an automatic blood coagulation analyzer “Coagrex-800” (available for Shimadzu Corp.). The measurements were conducted twice and the averages of the measurements were calculated. Lupus ratio (LR) of each sample was calculated according to the formula below (described in R. Schjetlein, et al., Thromb. Res. 1993, 69:239-250).

Lupus Ratio(LR)=(b/a)/(d/c)=b c/a d

wherein “a”, “b”, “c” and “d” are values measured under the conditions in Table 1.

TABLE 1

| | H-APTT | L-APTT |
|---|---|---|
| Mixture of patient's plasma and normal plasma | a | b |
| Pool of normal plasma | c | d |

The results for blood samples are shown in Table 2.

Clearly shown in Table 2, the coagulation times of abnormal plasma samples were prolonged comparing with the coagulation times of normal plasma samples. Furthermore, results indicate that LA-positive plasma samples treated with the L-APTT reagent have longer coagulation times than ones treated with the H-APTT reagent, while other abnormal plasma samples (i.e. samples of blood administered with warfarin or heparine) treated with the L-APTT reagent have similar coagulation times to ones treated with the H-APTT reagent.

TABLE 2

| | Coagulation time (sec.) | | | |
|---|---|---|---|---|
| SAMPLE | L-APTT | H-APTT | LR value | Decision |
| Normal plasma | 29.6 | 29.4 | 1.00 | − |
| Containing Heparin | 33.5 | 35.3 | 0.94 | − |
| Containing Warfarin 1 | 62.7 | 67.7 | 0.92 | − |
| Containing Warfarin 2 | 39.9 | 39.5 | 1.00 | − |
| Containing Warfarin 3 | 37.9 | 38.8 | 0.97 | − |
| Containing Warfarin 4 | 34.5 | 32.6 | 1.05 | − |
| LA positive 1 | 101.5 | 57.7 | 1.75 | + |
| LA positive 2 | 106.8 | 59.2 | 1.79 | + |
| LA positive 3 | 95.4 | 54.3 | 1.75 | + |
| LA positive 4 | 114.5 | 65.4 | 1.74 | + |

Samples were judged as positive (+) when the LR value of the sample is larger than 1.1, and judged as negative (−) when the LR value of the sample is not larger than 1.1. Clearly shown in Table 2, all samples of LA positive plasma were determined as positive(+), while the other abnormal plasma samples were determined as negative(−). Therefore, use of the inventive reagent kit is useful for easily and accurately distinguishing between blood samples from patients having disposition for LA-positive and other blood samples from individuals having disorders in blood coagulation.

### Comparative Example

The coagulation times of samples were measured according to LA test “Gradipore” (available for Medical Biological Laboratory). In this test, it is used the reagent kit which is a combination of a coagulation time reagent containing viper venom (hereinafter called as “RVVT reagent”) and a coagulation time reagent containing viper venom and soy-bean-derived phospholipid in an excessive amount (hereinafter called as “diluted RWT reagent”). As samples to be measured, the same kinds of samples as ones used in the above example were used. After measuring the coagulation times of samples, LR values were calculated in the same manner as the example. The results are shown in Table 3.

TABLE 3

| | Coagulation time (sec.) | | | |
|---|---|---|---|---|
| SAMPLE | d-RVVT | RVVT | LR value | Decision |
| Normal plasma | 36.8 | 31.4 | 1.00 | − |
| Containing Heparin | 56.4 | 30.2 | 1.59 | + |
| Containing Warfarin 1 | 116.9 | 65.1 | 1.53 | + |
| Containing Warfarin 2 | 59.8 | 38.9 | 1.31 | + |
| Containing Warfarin 3 | 57.1 | 36.5 | 1.33 | + |
| Containing Warfarin 4 | 61.3 | 35.7 | 1.47 | + |
| LA positive 1 | 74.1 | 35.5 | 1.78 | + |
| LA positive 2 | 81.4 | 36.6 | 1.90 | + |
| LA positive 3 | 62.3 | 33.2 | 1.60 | + |
| LA positive 4 | 81.2 | 38.7 | 1.79 | + |

As seen in Table 3, coagulation time of every abnormal plasma sample treated with the diluted RVVT reagent was prolonged comparing with one of a normal plasma sample treated with the same reagent. However, not only LA-positive plasma samples treated with the diluted RVVT reagent but also other abnormal blood plasma samples treated with the same showed longer coagulation times than samples treated with the RVVT reagent.

Accordingly the combinations of reagents having a different content of PS to the total content of phospholipids from each other may be effective for distinguishing between blood samples from LA-positive patients and blood samples having other disorders in blood coagulation.

This application is based on patent No. 2002-216782 filed on Japan, the contents of which hereby incorporated by reference.

What is claimed is:

1. A method for detecting lupus anticoagulant in blood comprising steps of:
comparing a first coagulation time measured by use of a first reagent containing phospholipids including phsophatidylserine and a second reagent containing calcium ions, with a second coagulation time measured by use of a third reagent containing phospholipids including phosphatidylserine and a fourth reagent containing calcium ions, and
detecting lupus anticoagulant in blood on the result obtained from the comparing step,
wherein the concentration of the phosphatidylserine in the first reagent ranges from 3 μg/ml to 1000 μg/ml;
the concentration of the phosphatidylserine in the second reagent ranges from 0.2 μg/ml to 200 μg/ml;
the content of phosphatidylserine to the total content of the phospholipids in the first reagent is different from the total content of the phospholipids in the second reagent; and
the concentration of the phosphatidylserine in the first reagent is higher than that of the phosphatidylserine in the second reagent.

2. The method of claim 1, wherein the phosphatidylserine is synthetic phosphatidylserine or at least 99% purified phosphatidylserine derived from natural resources.

3. The method of claim 1, wherein the concentration of the phosphatidylserine in the first reagent ranges from 30 μg/ml to 100 μg/ml

4. The method of claim 1, wherein the concentration of the phosphatidylserine in the third reagent ranges from 2 μg/ml to 20 μg/ml.

5. The method of claim 1, wherein each of the first and third reagents further contains phosphatidylethanolamine and phosphatidylcholine.

6. The method of claim 5, wherein the concentration of the phosphatidylethanolamine in each of the first and third reagents ranges from 0.1 μg/ml to 300 μg/ml, and the concentration of the phosphatidylcholine in each of the first and third reagents ranges from 2 μg/ml to 1000 μg/ml.

7. The method of claim 5, wherein the concentration of the phosphatidylethanolamine in each of the first and third reagents ranges from 1 μg/ml to 30 μg/ml, and the concentration of the phosphatidylcholine in each of the first and third reagents ranges from 20 μg/ml to 100 μg/ml.

8. The method of claim 1, wherein each of the first and third reagents further contains phosphatidylethanolamine, phosphatidylcholine and an activator.

9. The method of claim 8, wherein the activator is at least one selected from the group consisting of ellagic acid, kaolin, and celite.

10. The method of claim 1, wherein each of the first and third reagents further contains a viper venom.

11. The method of claim 1, wherein each of the first and third reagents further contains phosphatidylethanolamine, phosphatidylcholine, and viper venom.

12. The method of claim 10, wherein the viper venom is at least one selected from the group consisting of Russel's venom, textarin venom and ecarin venom.

13. The method of claim 1, wherein each of the first and third reagents further contains a tissue factor.

14. The method of claim 1, wherein each of the first and third reagents further contains phosphatidylethanolamine, phosphatidylcholine, and a tissue factor.

15. A method for detecting lupus anticoagulant in blood comprising steps of.
comparing a first coagulation time measured by use of a first reagent containing phospholipids including phosphatidylserine and a second reagent containing calcium ions, with a second coagulation time measured by use of a third reagent containing phospholipids including phosphatidylserine and a fourth reagent containing calcium ions; and
detecting lupus anticoagulant in blood on the result obtained from the comparing step,
wherein the concentration of the phosphatidylserine in the first reagent ranges from 3 μg/ml to 1000 μg/ml;
the concentration of the phsophatidylethanolamine in the first reagent ranges from 0.1 μg/ml to 300 μg/ml;
the concentration of the phsophatidylcholine in the first reagent ranges from 2 μg/ml to 1000 μg/ml;
the concentration of the phosphatidylserine in the second reagent ranges from 0.2 μg/ml to 200 μg/ml;
the concentration of the phsophatidylethanolamine in the second reagent ranges from 0.1 μg/ml to 300 μg/ml;
the concentration of the phsophatidylcholine in the second reagent ranges from 2 μg/ml to 1000 μg/ml;
the content of phosphatidylserine to the total content of the phsopholipids in the first reagent is different from the total content of the phospholipids in the second reagent; and
the concentration of the phosphatidylserine in the first reagent is higher than that of the phosphatidylserine in the second reagent.

16. The method of claim 15, wherein each of the first and third reagents further contains an activator.

17. The method of claim 15, wherein the concentration of the phosphatidylserine in the first reagent ranges from 30 μg/ml to 100 μg/ml and the concentration of the phosphatidylserine in the third reagent ranges from 2 μg/ml to 20 μg/ml.

18. The method of claim 15, wherein each of the first and third reagents further contains a viper venom.

19. The method of claim 15, wherein each of the first and third reagents further contains a tissue factor.

20. The method of claim 15, wherein the phosphatidylserine is synthetic phosphatidylserine or at least 99% purified phosphatidylserine derived from natural resources.
